(19) United States
(12) Patent Application Publication
(10) Pub. No.: US 2023/0176037 A1
(43) Pub. Date: Jun. 8, 2023

(54) COMPUTER-IMPLEMENTED METHOD AND APPARATUS FOR ASSOCIATING AN ODOR SUBSTANCE OF A GAS UNDER INVESTIGATION WITH AN ELECTRICAL SIGNAL

(71) Applicant: ZF Friedrichshafen AG, Friedrichshafen (DE)

(72) Inventor: Manuel FISCHER, Radolfzell (DE)

(73) Assignee: ZF Friedrichshafen AG, Friedrichshafen (DE)

(21) Appl. No.: 18/077,086

(22) Filed: Dec. 7, 2022

(30) Foreign Application Priority Data
Dec. 8, 2021 (DE) 102021213970.1

(51) Int. Cl. G01N 33/497; G01N 33/00; G01N 27/02; B60K 28/02

(52) U.S. Cl. CPC G01N 33/497; G01N 33/0031; G01N 27/02; B60K 28/02

(57) ABSTRACT

A computer-implemented method and device includes obtaining a first electrical signal on a basis of an interaction between one or more odor substances in a gas and at least one first odor sensor, filtering at least one predetermined odor group out of the gas and obtaining a filtered gas, wherein the odor group comprises at least one odor substance that is to be filtered out, obtaining a second electrical signal on the basis of an interaction between one or more odor substances in the filtered gas and the first odor sensor and/or at least one second odor sensor, determining a difference between the first and second electrical signals, and assigning this difference to the odor group.

### CROSS-REFERENCE TO RELATED APPLICATIONS

This application claims priority from German Application No. 102021213970.1, filed on Dec. 8, 2021, the entirety of which is hereby fully incorporated by reference herein.

### BACKGROUND AND SUMMARY

The present disclosure relates to a method and a device for assigning an electrical signal to an odor substance.

An odor can be identified as odor information (sensory information) by means of an odor sensor when one or more chemical (odor) substances act on odor cells in the odor sensor. Numerous odor sensors of the same type can be arranged in a sensor array for identifying odor information. An electronic signal resulting from the interaction between the chemical substances and the sensor array can be measured and evaluated using artificial intelligence.

There is a coating on the surfaces of the odor sensors that interacts with the chemical odor substances that cause the odor. It has not yet been possible to treat the surfaces such that only one specific gas or one specific odor substance interacts with the surface. A so-called cross-coupling takes place. Consequently, it is not possible to distinctly assign the signal that is measured to a specific odor substance. Moreover, the surfaces of identical types of odor sensors exhibit deviations that also have an effect on a precise assignment of the electrical signal to a gas or an odor substance. This is of particular relevance when the odor sensor is exposed to not just one specific substance, but instead is exposed to a mixture of gases, e.g. human exhalations.

In addition to the difficulty of measuring an electrical signal using an odor sensor, there is also the problem that an odor may be composed of numerous odor substances. There may be numerous similar to nearly identical odor substances, which generate identical or similar odors, although the electrical signals from these odor substances may differ.

EP 3 379 240 A1 describes an odor sensor that has at least two sensor elements, each of which has a substance-absorbing membrane for absorbing one or more odor substances in the air. The sensor elements also comprise a signal-converting unit for measuring the electrical properties of the substance-absorbing membrane after it has absorbed the substance. The substance-absorbing membrane has a main skeleton that contains an electrically conductive polymer and a doping agent for modifying the main skeleton of the electrically conductive polymer. The sensor elements each have substance-absorbing membranes with different portions of the main skeleton and the doping agent.

EP 3 187 852 A1 describes an odor identification system that comprises an operation assignment unit, which comprises at least two sensors that interact with substances that cause odors contained in an odor factor of a gas sample. The system also comprises a sensor data processing unit that processes data obtained from the interaction with the odor factor in the operation assignment unit, an odor factor information memory that stores the information for the odor factor and the interaction pattern information for the odor factor in advance, and a pattern recognition unit that identifies the odor factor on the basis of an interaction pattern by comparing the pattern processed by the sensor data processing unit and the information stored in the odor factor information memory.

It is therefore an object of the present disclosure to create a computer-implemented method and a device for assigning an electrical signal to at least one odor substance in a gas sample, which eliminates one or more the disadvantages described above. In particular, an object of the present disclosure is to determine the presence of a specific gas or at least a specific odor group.

The first aspect of this problem is solved by a computer-implemented method for assigning an electrical signal to at least one odor substance in a gas sample. The method comprises obtaining a first electrical signal on the basis of an interaction of one or more odor substances in the gas with at least one first odor sensor. At least one predetermined odor group is filtered out of the gas sample, such that a gas is obtained in which at least one of the odor substances that is to be filtered out is contained in the odor group. The method also comprises obtaining a second electrical signal on the basis of an interaction between one or more of the odor substances in the filtered gas and the first odor sensor and/or at least one second odor sensor. A difference between the first and second electrical signals is determined and this difference is then assigned to odor group.

According to the method proposed herein, an odor group is filtered out and the signal difference is assigned to this odor group. When filtering out the odor group, one or more predetermined odor substances and odor substances with similar behaviors can be filtered out of the gas sample, such that the signal difference can be assigned to the odor group. The odor substance that is to be filtered out and the odor substance in similar odor substances can be filtered out with the proposed method. These can contain numerous substances and/or compounds. The resulting signal difference can represent the filtered odor substance and the similar odor substances, thus correcting possible imprecisions occurring in the prior art. This results in a better identification of the odor group. Consequently, it is not absolutely necessary to treat a surface of the odor sensor such that it can only interact with one specific odor substance. Furthermore, a gas containing not only this one specific odor substance can be used.

The first electrical signal can be obtained with at least one first odor sensor, and/or the second electrical signal can be obtained with the first and/or at least one second odor sensor.

The filtering can take place using a method or filtering process according to the “highly selective gas sensing enabled by filters” by Jan van den Broek, Ines c. Weber, Andreas T. Güntner and Sotiris E. Pratsinis, published on Nov. 12, 2020 by the Royal Society of Chemistry.

The signal difference can be obtained by subtracting the first signal from the second signal or vice versa. Alternatively, the signal difference can be obtained by comparing the first and second signals. In particular, the signal difference can be an n-dimensional analysis or subtraction of the two signals. An odor signal can have the form of a two-dimensional signal, comprising frequency and amplitude, such that a two dimensional analysis of the signal or signal difference can be made. Other information can also be contained in the signal, such that a three dimensional or multidimensional analysis can be made.

The odor group can comprise two or more odor substances. An odor substance can be in the form of a gas and/or a liquid. The odor substance can also contain one or more substances, compounds, and/or molecules or molecular compounds.

The method can also comprise creating an odor database that contains at least one entry that characterizes at least the odor group. The method can also comprise processing the entry on the basis of the odor group and the signal difference assigned thereto, such that the entry in the database characterizes the odor group and the signal difference assigned thereto.

The entry in the odor database can also characterize an odor and/or a classification of the odor group in which the classification indicates at least an illness, a hazardous substance, a poisonous (toxic) substance, a substance indicating putrefaction, a substance indicating burning, a psychotropic substance and/or a drug in the odor group that alters a physical state and/or alters consciousness and/or perception. These psychotropic substances or drugs can comprise any of the prevalent psychotropic drugs, alcohol, cannabis, or other illegalized substances. Illnesses can result in a change in the odor substances in human exhalations, such that the identification of odor substances and/or concentrations of these substances can indicate the presence of an illness. Furthermore, illnesses are spread by certain bacteria and viruses that contain specific substances that can interact with the surfaces of odor sensors, or can be filtered out. Substances indicating putrefaction occur in food such as fruit, vegetables, meat and fish, for example, due to decomposition caused by microorganisms and comprise gases or substances that characterize fermentation of the foodstuff. Substances indicating burning can indicate or comprise an aerosol resulting from burning in a finely distributed form composed of dust particles (soot, flue ash, unburnt elements) and liquid droplets (water, oil vapors, acid vapors, liquid burning residues). The hazardous substances can be substances that dissolve in a liquid film in the airway resulting in chemical inflammation (ranging from eye irritation causing teardrops to toxic pulmonary edema), gases that act as systemic respiratory poisons by suppressing oxygen transport by hemoglobin (CO) or cellular utilization of O₂ by the mitochondria, and nitrogen gases, which can lead to hypoxia in sufficient concentrations through the lowering of the partial pressure of oxygen. The hazardous substances can also contain carbon dioxide (CO₂), which has a narcotic effect. A poisonous substance (a poison), also referred to as a toxicant, is a substance that can harm life forms, in particular humans in this case, through its metabolic processes, by invading the organism, even to a very low extent. As exposure to a substance increases, there is a greater probability that this will cause harm to the health of the organism by poisoning it.

The filtering can comprise a filtering of the odor group through sorption, size-specific filtering, and catalytic filtering. The filtering can also comprise filtering by means of an electric field, mechanical and/or physical separation, and/or chemical conversion and/or a frequency for filtering the odor group. The filtering can also comprise filtering by accelerating the gas or individual components of the gas such as the odor substances in the odor group, and deflecting these components.

Odor substances can be filtered out with size-specific filtering such that only the odor substances of a predefined size or smaller can pass through the filter. Sorption filtering results in certain molecules and/or molecular compounds of odor substances binding to the surface of the filter, such that they can be filtered out of the gas.

The odor database entry can comprise filter information that characterizes the filtering of the odor group.

The method can also comprise determining the concentration of the odor group in the gas sample on the basis of the first signal, the second signal, the signal difference, the database entry, and/or the designated odor group.

The method can also comprise issuing a warning signal if the odor group is classified as a psychotropic substance and/or drug, and/or if the concentration of the odor group exceeds a predetermined concentration threshold value.

The steps for determining the signal difference, processing the odor database entry, determining the concentration and outputting a warning signal can take place using an odor algorithm. The odor algorithm can be a machine learning algorithm, or it can comprise such. A second aspect of the object is achieved with an odor data base, which is obtained using the method for the first aspect.

A third aspect of the object is achieved with a computer program for assigning an electrical signal to at least one odor substance in a gas, comprising commands with which a processor executes the method in the first aspect when this program is executed by the processor.

A fourth aspect of the object is achieved by a device for assigning an electrical signal to at least one odor substance in a gas sample. The device comprises at least one first odor sensor that is configured to obtain a first electrical signal on the basis of an interaction between one or more odor substances in the gas sample and the first odor sensor, and at least one filter that is configured to filter at least one predetermined odor group out of the gas sample and to obtain a filtered gas in which the odor group comprises at least one odor substance that is to be filtered out. The first odor sensor and/or at least one second odor sensor in the device are configured to obtain a second electrical signal on the basis of the interaction between one or more odor substances in the filtered gas and the first and/or second odor sensor. The device also comprises a memory and a processor that is configured to determine a signal difference between the first and second electrical signals and to assign the signal difference to the odor group.

The device according to the fourth aspect and the method according to the first aspect can be used in buildings, reservoirs, containers, rooms, or, as described below, in a vehicle. Odors relating to safety can be detected in buildings with the device and method proposed herein, such that a warning signal or alarm can be triggered if such an odor is detected, and/or a concentration of the odor in question exceeds a predetermined concentration in the air. Containers are used for transporting fruit over greater distances, for example. The fruit is sealed in the container during transport, and normally cannot be inspected visually from the exterior. Gases and odors can result from mold and decomposition of the substances caused by microorganisms, which can indicate rotting or putrefaction of the fruit. The device can be placed inside the container, and transmit signals to a receiver outside the container. By this means it is possible to check on the fruit on a continuous basis.

The memory can also be configured to create an odor database that contains at least one entry in which at least the odor group is characterized. The processor can also be configured to process the entry on the basis of the odor group and the signal difference assigned thereto, such that the entry characterizes the odor group and the designated signal difference.

The entry in the odor database can also characterize an odor and/or a classification of the odor group in which the classification indicates a psychotropic substance and/or drug in the odor group that alters a physical state and/or alters consciousness and/or perception.

The filter can be a sorption filter, size-specific filter and/or catalytic filter. The filter can also be configured to filter by means of an electric field, a mechanical and/or physical separation and/or chemical conversion for filtering the odor group, and/or a frequency for filtering the odor group. The filter can also be configured to filter by accelerating the gas or individual components of the gas such as odor substances, and to deflect these components.

The processor can also be configured to determine a concentration of the odor group in the gas sample on the basis of the first odor signal, the second odor signal, the signal difference, and/or the designated odor group.

The processor can also be configured to issue a warning signal if the odor group is classified as a psychotropic substance and/or drug, and/or if the concentration of the odor group exceeds a predetermined concentration threshold value.

The device can also comprise a transceiver that is configured to transmit and/or receive data, in particular from the first odor sensor, the second odor sensor, the filter, and/or the processor. The transceiver can also be configured to receive and/or transmit data from and to external units, in particular by means of a wireless network. These data can characterize the odor database and/or the filter information.

A fifth aspect of the object is achieved by a vehicle that comprises a device according to the fourth aspect.

The processor can also be configured to determine who is operating the vehicle and require that the operator submit breath sample. The first odor sensor can be configured to obtain a first electric signal from the breath sample on the basis of an interaction between one or more odor substances in the breath sample and the first odor sensor. The filter can also be configured to filter at least one safety odor group out of the breath sample in which the safety odor group comprises at least one substance indicating an illness, a psychotropic substance, and/or a drug. The first odor sensor and/or the second odor sensor can be configured to obtain a second electrical signal from the breath sample on the basis of an interaction between one or more odor substances in the filtered breath sample and the first and/or second odor sensor. The processor can also be configured to determine a difference between first and second electrical breath sample signals, such that if there is such a difference in the signals, this signal difference is assigned to the safety odor group. A difference in the breath sample signals can be established if there is a difference in the signals from the breath samples or a predetermined threshold value has been exceeded. The predetermined threshold value can be selected such that noise and/or errors in the measurement of the odor signals is suppressed when determining if there is a difference in the breath sample signals. As a result, an incorrect diagnosis due to noise and/or measurement error can be prevented. The processor can also be configured to issue a vehicle ignition lock signal to temporarily prevent operation of the vehicle on the basis of a safety restriction that is satisfied if the signal difference in the breath samples is that assigned to the safety odor group. The safety restriction can also be satisfied if a concentration of the safety odor group in the breath sample exceeds the predetermined concentration threshold value. The concentration of the safety odor group can be determined in the breath sample on the basis of the first breath sample signal, the second breath sample signal, the breath sample signal difference, and/or the designated safety odor group.

Accordingly, a driver can be checked prior to driving the vehicle according to objective standards, thus increasing the safety for the driver and other road users. Furthermore, a subjective observation of the driver could result in an incorrect evaluation, e.g. in evaluating the alcohol level. The device provides the driver with a possibility for checking this.

The first odor sensor can be configured to obtain a first electrical signal from ambient air on the basis of an interaction between one or more odor substances in the ambient air around the driver and/or in the vehicle and the first odor sensor. The ambient air can be the air inside and/or outside the vehicle interior, in particular outside the vehicle. The filter can be configured to filter at least one poison odor group out of the ambient air and obtain a filtered ambient air in which the poison odor group comprises at least a poisonous substance, a hazardous substance, a substance indicating putrefaction, and/or at least a substance indicating burning. The poisonous substances can be exhaust fumes from the vehicle or at least one other vehicle, or other substances that are toxic to humans. The first odor sensor and/or second odor sensor can be configured to obtain a second electrical ambient air signal on the basis of an interaction between one or more odor substances in the filtered ambient air and the first and/or second odor sensor. The processor can also be configured to determine a signal difference between the first and second electrical ambient air signals, such that if there is a difference in the ambient air signals, the signal difference is assigned to the poisonous odor group, and a warning signal is issued to indicate the presence of danger to the driver on the basis of poison safety restriction that has been satisfied. The poison safety restriction is satisfied if the difference in the ambient air signals is that assigned to the poisonous odor group, and/or the poison safety restriction is satisfied if a concentration of the poisonous odor group in the ambient air exceeds a predetermined concentration threshold value. A difference in the ambient air signals can be determined in the same manner as the difference in breath sample signals, in which the ambient air signals exhibit a difference, or a predetermined threshold value is exceeded. In this manner, the operator's environment can be checked for the poisonous odor group. By way of example, the vehicle may be caught in traffic in a tunnel. If the exhaust fumes from the vehicles exceed a predetermined concentration in the ambient air, the vehicle can then inform the driver of these exhaust fumes, and the driver can close the windows. If the vehicle catches on fire, or there is a fire in the vicinity thereof, the driver can be informed thereof by the proposed device.

Explanations and features referring to the method and the device can also comprise features of the vehicle. Furthermore, explanations and features relating to the method can also comprise features of the device.

Preferred exemplary embodiments shall be explained below in reference to the drawings.

### BRIEF DESCRIPTION OF THE DRAWINGS

FIG. 1 shows a schematic illustration of a device for assigning an electrical signal to at least one odor substance in a gas sample;

FIG. 2 shows a schematic illustration of how the first and second odor signals are obtained; and

FIG. 3 shows a schematic illustration of how an electrical signal is assigned to at least one odor substance in a gas sample.

### DETAILED DESCRIPTION

Identical elements or elements that substantially have the same or similar functions have the same reference symbols in the figures.

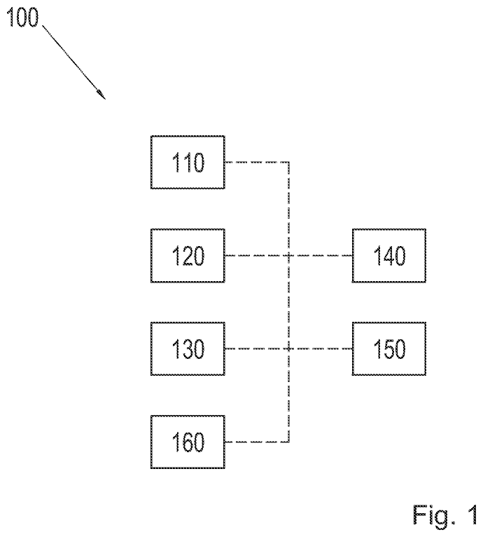

FIG. 1 shows a device 100 for assigning an electrical signal to at least one odor substance in a gas sample. The device 100 comprises a first odor sensor 110, a filter 120, a second odor sensor 130, a memory 140, a processor 150 and a transceiver 160. The units in the device 100 are coupled to one another for signal transfer.

The first and second odor sensors 110, 130 are configured to generate a first and second electrical signal on the basis of an interaction with one or more odor substances in a gas. The odor sensors 110, 130 have surfaces for this that have been treated such that substances and/or compounds that the odor substances are composed of interact with the surfaces, e.g. binding to the surfaces at the molecular level. The electrical signal is altered by the interactions with the respective surfaces of the odor sensors 110, 130. The electrical signals can characterize a first state, in which the surfaces do not interact with the odor substances, and a second state, in which the odor substances do interact with the surfaces. The progressions of the electrical signals can also be plotted over time.

The filter 120 is configured to filter out a predetermined odor group from a gas and obtain a filtered gas, wherein the odor group comprises at least one odor substance that is to be filtered out. By way of example, the filter can contain a filter membrane with a predetermined minimum pore size. The gas can contain various gas components, or odor substances, of different sizes. When the gas is filtered through the filter 120, those odor substances that are larger than the pores in the filter are filtered out of the gas by the filter membrane. The filtered gas then contains fewer odor substances than the original gas. An odor database containing at least one entry is stored in the memory 140, in which at least one odor group is characterized. The characterized odor group corresponds to the at least one odor group that has been filtered out of the gas. The odor database can also contain filter information representing the characteristics of the filter, e.g. the pore sizes of the filter membrane.

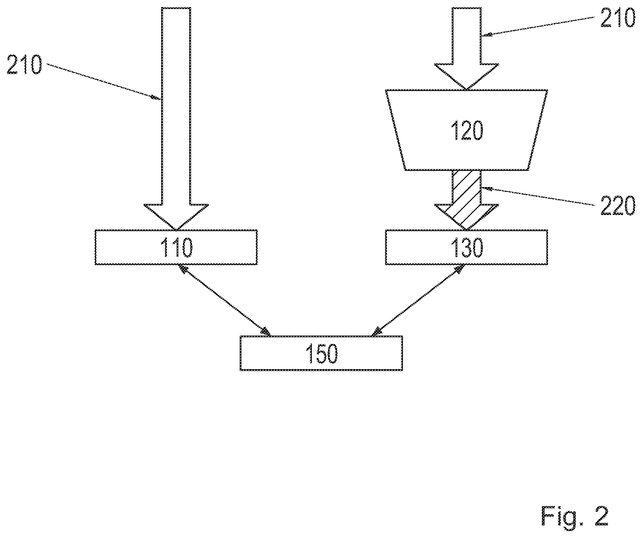

The transceiver 160 is configured to receive data from the various units in the device 100. The transceiver 160 is also configured to transmit or receive data to or from external units. As FIG. 2 shows, the first odor sensor 110 generates a first electrical signal on the basis of an interaction between one or more odor substances in a gas 210 that is to be examined and the first odor sensor 110, in particular the treated surface of the first odor sensor 110.

The gas 210 that is to be examined is also filtered with the filter 120 such that at least one predetermined odor group 210 is filtered out of the gas 210, wherein the predetermined odor group comprises at least one predetermined odor substance. A filtered gas 220 is obtained with the filter 120 that is then sent to the second odor sensor 130. This filtered gas 220 can also be sent to the first odor sensor 110.

A second electrical signal is generated on the basis of the interaction between the filtered gas 220 and the second odor sensor 130. The first and second odor signals are then sent to the processor 150.

The processor 150 is configured to determine a difference between the first and second odor signals. This can take place using an odor signal algorithm, which is a machine learning algorithm in this case. The difference between the odor signals is obtained by subtracting the second signal from first. The difference can then be assigned to the odor group that has been filtered out. The entry in the odor database in the memory 140 is then supplemented and/or modified with regard to this odor group by the signal difference.

The device 100 can be used in or with a vehicle, as explained below. It can also be used in buildings, spaces, vessels, outdoors, and in transporters, such as containers.

According to a further aspect of the present disclosure, the device 100 is contained in a vehicle. The vehicle can be a passenger automobile, truck, bus, scooter, motorcycle, ship, airplane, or an at least partially autonomous vehicle in particular. The vehicle should only be operated by a driver who is subjectively and objectively able to do so. Objective measures comprise, among other things, an alcohol level, which should/must be below a predetermined threshold value. Vehicles also should not be operated after consuming psychotropic substances and/or drugs that alter a physical state and/or consciousness and/or perception, which can also be determined objectively. These objective measures may also be subject to guidelines and legal provisions. Consequently, it may be the case with vehicles that the operator is not allowed to operate a vehicle if evidence of any such substance is detected, or an alcohol level is not below the threshold level.

In order to increase the safety for the vehicle operator, other potential passengers, as well as other road users, a vehicle is equipped with the device 100 in this aspect of the present disclosure.

The processor 150 is configured to determine who is operating the vehicle and require a breath sample 210 from the operator. The breath sample 210 can be obtained using a mouthpiece provided for this, with which the submitted breath sample 210 is conducted to the first and second odor sensors 110, 130. Air can also be sampled from inside the vehicle and conducted to the odor sensors 110, 130. A portion of this air can also comprise a breath sample from the operator. The first odor sensor 110 is configured to obtain a first electrical breath sample signal on the basis of an interaction between one or more odor substances in the breath sample 210 and the first odor sensor 110. At least one safety odor group is also filtered out of the breath sample by the filter 120, which contains at least one safety odor substance classified as a substance indicating illness, psychotropic substances and/or drugs. A filtered breath sample 220 is obtained with the filter 120. The second odor sensor 130 is configured to obtain a second electrical breath sample signal on the basis of an interaction between one or more odor substances in the filtered breath sample 220 and the second odor sensor 130. The processor 150 determines a difference between the first and second breath sample signals. If the processor 150 determines that there is a difference in the breath sample signals, this difference is assigned to the safety odor group. This can be established if there is a difference in the breath sample signals, or a predetermined threshold value is exceeded. The predetermined threshold value can be selected such that any noise and/or errors in the measurements of the odor signals are suppressed when determining a difference in the breath sample signals. This prevents an inaccurate diagnosis caused by noise and/or errors. Consequently, it may be determined that there is an odor substance in the breath sample that can have an effect on the driving abilities of the vehicle operator. The processor then issues an ignition lock signal that temporarily prevents the vehicle from being operated on the basis of a safety restriction having been satisfied. The safety restriction is satisfied if the difference in the signals from the breath sample is that assigned to the safety odor group. The safety restriction can also be satisfied if a concentration of the safety odor group in the breath sample 210 exceeds the predetermined concentration threshold value. One example of this is when the alcohol concentration in the breath sample 210 exceeds a predetermined concentration threshold level. Consequently, operation of the vehicle can be prevented in accordance with an objective measure for when an operator is not fit to operate a vehicle, thus increasing traffic safety.

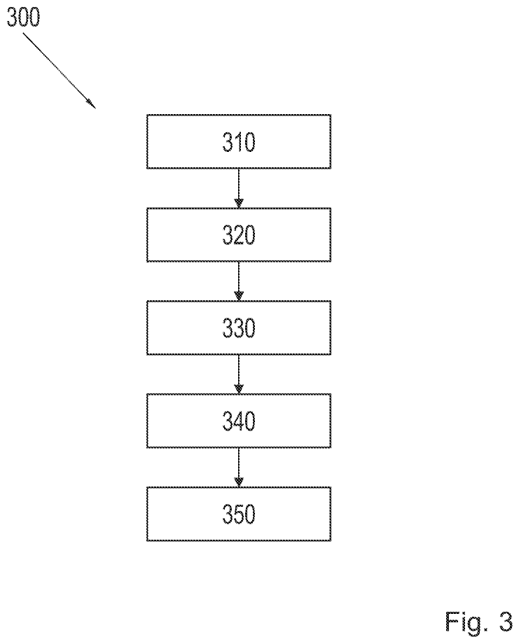

FIG. 3 shows a schematic illustration of a method 300 for assigning an electrical signal to at least one odor substance in a gas 210 sample. The method 300 comprises a step 310 for obtaining a first electrical signal on the basis of an interaction between one or more odor substances in the gas 210 sample and at least one first odor sensor 110. The method 300 also comprises a step 320 for filtering at least one predetermined odor group out of the gas 210 sample with a filter, and obtaining a filtered gas 220, wherein the odor group comprises at least one odor substance that is to be filtered out. A second electrical signal is obtained in a third step 330 on the basis of an interaction between one or more odor substances in the filtered gas 220 and the first odor sensor 110 and/or at least one second odor sensor 130. The method also comprises a step 340 for determining a difference in the first and second electrical odor signals, and a step 350 for assigning this signal difference to the odor group.

### REFERENCE SYMBOLS

100 device for assigning an electrical signal to at least one odor substance in a gas sample
110 first odor sensor
120 filter
130 second odor sensor
140 memory
150 processor
160 transceiver
210 gas
220 filtered gas
300 method for assigning an electrical signal to at least one odor substance in a gas sample
310 obtaining a first electrical signal
320 filtering at least one predetermined odor group out of the gas sample
330 obtaining a second electrical signal
340 determining a difference in the first and second electrical signals
350 assigning the signal difference to the odor group

1. A computer-implemented method comprising:
obtaining a first electrical signal on a basis of an interaction between one or more odor substances in a gas sample and at least one first odor sensor;
filtering at least one predetermined odor group out of the gas sample and obtaining a filtered gas, wherein the odor group comprises at least one odor substance that is to be filtered out;
obtaining a second electrical signal on a basis of an interaction between one or more odor substances in the filtered gas and the first odor sensor and/or at least one second odor sensor;
determining an odor signal difference in the first and second electrical signals; and
assigning the odor signal difference to the odor group.

2. The method according to claim 1, further comprising:
creating an odor database that contains at least one entry, wherein the at least one entry characterizes at least the odor group; and
processing the at least one entry on a basis of the odor group and the signal difference assigned thereto, such that the at least one entry characterizes the odor group and the signal difference assigned thereto.

3. The method according to claim 2, wherein
the at least one entry in the odor database also characterizes an odor and/or a classification of the odor group,
wherein the classification indicates at least one of an illness, a hazardous substance, a poisonous substance, a substance indicating putrefaction, substances indicating burning, a psychotropic substance and/or a drug in the odor group that alters a physical state, consciousness, and/or perception of a user.

4. The method according to claim 1, wherein the filtering comprises filtering with a sorption filter, a size-specific filter, or a catalytic filter for filtering out the odor group, and/or
wherein the filtering comprises filtering with an electrical field, a mechanical and/or physical separation, and/or chemical conversion, for filtering out the odor group.

5. The method according to claim 1, further comprising:
determining a concentration of the odor group in the gas sample on a basis of the first electrical signal, the second electrical signal, the difference in the first and second electrical signals, the entry in the odor database, and/or the designated odor group.

6. The method according to claim 1, further comprising:
issuing a warning signal in response to the odor group being classified as a psychotropic substance and/or a drug, and/or in response to a concentration of the odor group exceeding a predetermined concentration threshold value.

7. A non-transitory computer-readable medium having stored thereon a program that, when executed by a computer device, cause the computer device to perform a method comprising:
obtaining a first electrical signal on a basis of an interaction between one or more odor substances in a gas sample and at least one first odor sensor;
obtaining a second electrical signal on a basis of an interaction between one or more odor substances in a filtered gas and the first odor sensor and/or at least one second odor sensor, wherein the filtered gas is obtained by filtering at least one predetermined odor group out of the gas sample and wherein the odor group comprises at least one odor substance that is to be filtered out;
determining an odor signal difference in the first and second electrical signals; and
assigning the odor signal difference to the odor group

8. A device for assigning an electrical signal to at least one odor substance in a gas sample, comprising:
at least one first odor sensor configured to obtain a first electrical signal on a basis of an interaction between one or more odor substances in a gas sample and the first odor sensor;
at least one filter configured to filter at least one predetermined odor group out of the gas sample and obtain a filtered gas, wherein the odor group comprises at least one odor substance that is to be filtered out;
wherein the first odor sensor and/or at least one second odor sensor are configured to obtain a second electrical signal on a basis of an interaction between one or more odor substances in the filtered gas and the first and/or second odor sensor; and
a processor configured to determine a difference in the first and second electrical signals, and assign the signal difference to the odor group.

9. The device according to claim 8, further comprising:
a memory configured to store an odor database that contains at least one entry, wherein the at least one entry characterizes at least the odor group,
wherein the processor is also configured to process the at least one entry in the odor database on a basis of the odor group and the difference in the first and second electrical odor signals assigned thereto, such that the entry characterizes the odor group and the signal difference assigned thereto.

10. The device according to claim 9, wherein the at least one entry in the odor database also characterizes an odor and/or a classification of the odor group,
wherein the classification indicates at least one of an illness, a hazardous substance, a poisonous substance, a substance indicating putrefaction, a substance indicating burning, a psychotropic substance and/or a drug in the odor group which alters a physical state, a consciousness, and/or a perception of a user.

11. The device according to claim 8, wherein the filter is a sorption filter, a size-specific filter, and/or a catalytic filter, and/or
wherein the filter is configured to filter by means of an electrical filed, a mechanical and/or physical separation, and/or chemical conversion, for filtering out the odor group.

12. The device according to claim 8, wherein the processor is further configured to determine a concentration of the odor group in the gas sample on a basis of the first electrical signal, the second electrical signal, the difference in the first and second electrical signals, and/or the designated odor group.

13. The device according to claim 8, wherein the processor is further configured to issue a warning signal in response to the odor group being classified as a psychotropic substance and/or a drug, and/or in response to a concentration of the odor group exceeding a predetermined concentration threshold value.

14. A vehicle comprising the device according to claim 8.

15. The vehicle according to claim 14, wherein the processor is further configured to:
determine an operator operating the vehicle; and
require a breath sample from the operator,
wherein the first odor sensor is configured to obtain the first electrical breath sample signal for the breath sample on a basis of an interaction between one or more odor substances in the breath sample and the first odor sensor,
wherein the filter is configured to filter at least one safety odor group out of the breath sample and obtain a filtered breath sample,
wherein the safety odor group comprises at least one substance that indicates an illness, a psychotropic substance, and/or a drug,
wherein the first odor sensor and/or the second odor sensor are configured to obtain a second electrical breath sample signal for the breath sample on a basis of an interaction between one or more odor substances in the filtered breath sample and the first and/or second odor sensor,
wherein the processor is also configured to:
determine a difference between the first and second electrical breath sample signals, and if there is a difference in the signals, assign this difference to the safety odor group, and
issue an ignition lock signal to temporarily prevent operation of the vehicle on a basis of a safety restriction being satisfied,
wherein the safety restriction is satisfied in response to the difference in the first and second electrical breath sample signals being assigned to the safety odor group, and/or
wherein the safety restriction is satisfied in response to a concentration of the safety odor group in the breath sample exceeding the predetermined concentration threshold value.

16. The vehicle according to claim 14,
wherein the first odor sensor is configured to obtain a first electrical ambient air signal for the ambient air on a basis of an interaction between one or more odor substances in the ambient air of the operator and/or the vehicle with the first odor sensor,
wherein the filter is configured to filter at least one poisonous odor group out of the ambient air and obtain a filtered ambient air,
wherein the poisonous odor group comprises at least a poisonous substance, a hazardous substance, a substance indicating putrefaction, and/or at least a substance indicating burning,
wherein the first odor sensor and/or the second odor sensor are configured to obtain a second electrical ambient air signal for the ambient air on a basis of an interaction between one or more odor substances in the filtered ambient air and the first and/or second odor sensor,
wherein the processor is also configured to:
determine a difference between the first and second electrical ambient air signals;
assign the difference between the first and second electrical ambient air signals to the poisonous odor group; and
issue a warning signal indicating a danger to the driver on a basis of a poison safety restriction being satisfied,
wherein the poison safety restriction is satisfied in response to the difference between the first and second electrical ambient air signals being assigned to the poisonous odor group, and/or
wherein the poison safety restriction is satisfied in response to a concentration of the poisonous odor group in the ambient air exceeding a predetermined concentration threshold value.
